(19) United States
(12) Patent Application Publication
(10) Pub. No.: US 2008/0305175 A1
(43) Pub. Date: Dec. 11, 2008

(54) Micro-Container

(75) Inventors: Yubo Wang, SINGAPORE (SG); Wei Zheng, Singapore (SG); Mingyong Han, Singapore (SG); Kaiyu Wang, Singapore (SG)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AGENCY FOR SCIENCE, TECHNOLOGY AND RESEARCH, SINGAPORE (SG)

(21) Appl. No.: 11/947,522

(22) Filed: Nov. 29, 2007

Related U.S. Application Data
(60) Provisional application No. 60/867,974, filed on Nov. 30, 2006.

(51) Int. Cl. A61K 9/14; C08J 9/28; C08J 9/00; H01B 1/12; B01J 31/06

(52) U.S. Cl. 424/501; 521/64; 521/50; 252/500; 502/159

(57) ABSTRACT

There is disclosed a method of making a micro-container. The method comprises the step of evaporating a swelling agent solution absorbed in a polymer micro-particle to form an inner void therein. The evaporating step is undertaken under conditions to form a conduit extending through the shell wall of said micro-particle and into the inner void.

### TECHNICAL FIELD

The present invention generally relates to a micro-container and a method of making the same, such as those that can be used to contain substances such as drugs.

### BACKGROUND

Polymer microspheres have been employed as micro-carriers or capsules for containing substances such as drugs. Due to their relatively small size, polymer microspheres can greatly improve the delivery and performance of drugs by controlled and targeted delivery of drugs to the active site of a patient's body.

Polymer microspheres are usually formed during the process of physical encapsulation of drugs with preformed polymers. However, this often leads to a series of problems in the polymer microspheres due to their weak strength and broad size distribution. Hence, this may result in too rapid drug dumping and incongruous release behavior of the drug.

Consequently, an increased interest of producing hollow microspheres has been contemplated due to their good shell strength and narrow size distribution, which are desirable characteristics for drug carriers. Furthermore, hollow polymer microspheres have gained importance in the pharmaceutical industry as both drug encapsulants and vehicles of drug carriage, in which the active agent is either protected during its passage through the body or in storage until its release, or undergoes controlled release within the body.

Other attractive characteristics of hollow polymer microspheres include thermal resistance, low density, thermal insulation, and optical opacity, which have enabled them to be used as carrier devices in other industries such as the paint, ink, paper and cosmetics.

One problem with synthesizing hollow polymer microspheres are the synthetic conditions employed during their formation, which can make in-situ encapsulation of sensitive materials impossible. Furthermore, the physical incorporation or loading of the materials into preformed hollow polymer microspheres, and subsequent release of the materials can be difficult due to the low permeability of the shells walls of the microspheres.

One method of preparing hollow microspheres (e.g. hollow polystyrene microspheres) employs a dynamic swelling technique, involving phase separation in the presence of a cross-linking agent by seeded polymerization. This two-step polymerization process requires that a polystyrene seed be first dispersed in an ethanol or water mixture in which a cross-linking agent, a stabilizer and an organic solvent such as toluene are dissolved. Water is added continuously to the system to allow the cross-linking agent and the organic solvent to be absorbed by the polystyrene seed before polymerization is carried out. As polymerization proceeds, the polystyrene moves towards the interior surface of the particles due to the cross-linking reaction, thereby allowing the hydrophobic organic solvent to separate in the center of the particles. As a result, hollow particles are obtained after the organic solvent is removed. The size of the core can be controlled by varying the degree of swelling, or by the types of organic solvents used.

The hollow polymer (polystyrene) microspheres that are used to encapsulate a drug or substance typically require precise control over their sphere size and shell thickness. With such precise control, the drug or substance delivery kinetics, such as the rate at which a drug or substance is released by the microspheres, can then be controlled. However, due to the low permeability of the hollow polymer microspheres' shell walls, the loading or release of substances into or from pre-formed hollow polymer microspheres can be difficult. To solve this problem, hollow microspheres have been designed to increase their shell permeability by altering controlled conditions, such as temperature, pH, ion presence or ionic strength etc. As a result of such external stimuli, the loading or release conditions and the resulting morphological change of the microspheres may have undesirable effects on the loaded substances.

There is a need to provide hollow polymer microspheres for encapsulation of various substances that would overcome, or at least ameliorate, one or more of the disadvantages described above.

There is a need to provide an improved method for controlled loading and release of materials in hollow polymer microspheres.

### SUMMARY OF INVENTION

According to a first aspect there is provided a method of making a micro-container comprising the step of evaporating a swelling agent solution absorbed in a polymer micro-particle to form an inner void therein, wherein said evaporating is undertaken under conditions to form a conduit extending through the shell wall of said micro-particle and into the inner void.

Advantageously, the evaporation conditions are selected to create the conduit extending through the wall of the micro-particle due to release of relatively high pressure vaporized swelling agent bursting through the weakest point on the shell wall of the micro-particle.

According to a second aspect there is provided a method of making a micro-container comprising the steps of:

polymerizing, in droplet form, a monomeric mixture comprising a swelling agent solution to thereby form a polymer micro-particle having the swelling agent solution absorbed therein; and

evaporating said swelling agent solution from said micro-particle to form an inner void therein, wherein said evaporating is undertaken under conditions to form a conduit extending through the shell wall of the micro-particle and into the inner void.

According to a third aspect there is provided a micro-container comprising a polymer micro-particle having an inner void and a conduit extending through the shell wall of the micro-particle to said inner void.

According to a fourth aspect there is provided a micro-container for drug delivery comprising:

a polymer micro-particle having an inner void and a conduit extending through the shell wall of the micro-particle to said inner void; and

a drug loaded within said inner void.

According to a fifth aspect there is provided a method of delivering a drug to a patient comprising the step of administering the micro-container as defined in the fourth aspect to the patient.

### DEFINITIONS

The following words and terms used herein shall have the meaning indicated:

The term “biocompatible” as used herein refers to materials that do not elicit a substantial detrimental response in vivo.

The term “biodegradable polymers” as used herein refers to polymers that degrade fully (i.e. down to monomeric species) under physiological or endosomal conditions. Biodegradable polymers are not necessarily hydrolytically degradable and may require enzymatic action to fully degrade.

The term “micro-particle” is to be interpreted broadly to, unless specified, relate to an average particle size of between about 0.5 μm to about 100 μm. In embodiments where the particles are substantially spherical, the particle size may refer to the diameter of the particles. In embodiments where the particles are non-spherical, the particle size range may refer to the equivalent diameter of the particles relative to spherical particles.

The term “pinhole” as used herein refers to one embodiment of a conduit extending through the shell wall of a micro-particle.

The term “spheres” as used herein refers to an approximate sphere-shaped micro-container and includes all natural spheres which may be true spheres, oblate, prolate and the like.

The term “substantially” as used herein does not exclude “completely” e.g. X which is “substantially absorbed” by Y may be completely absorbed by Y. Where necessary, the word “substantially” may be omitted from the definition of the invention.

The term “swelling agent solution” as used herein refers to a liquid that interacts with a material (e.g. seed particles) and causes such material to undergo a volumetric expansion.

The term “swelling ratio” as used herein refers to the ratio of the volume of n-hexane to the mass of seed particles.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of a micro-container and a method of making the same will now be disclosed.

The disclosed embodiments describe a method of making a micro-container comprising the step of evaporating a swelling agent solution absorbed in a polymer micro-particle to form an inner void therein, wherein said evaporating is undertaken under conditions to form a conduit extending through the shell wall of said micro-particle and into the inner void.

The micro-particle may be in the form of a microsphere.

Advantageously, the evaporation conditions are to create the conduit extending through the wall of the microsphere due to release of the relatively high pressure vaporized swelling agent bursting through the weakest point on the shell wall of the microsphere.

The swelling agent solution may be an organic liquid. In one embodiment, the swelling agent solution is a non-polar hydrocarbon liquid. The non-polar hydrocarbon liquid may be a saturated or unsaturated with about 4 to about 8 carbon atoms. The saturated non-polar hydrocarbon liquid may be selected from the group consisting of n-pentane, n-hexane, n-heptane, n-octane. In one embodiment, the swelling agent solution is n-hexane.

The evaporation step may be undertaken at a temperature less than the boiling point of the swelling agent solution. In one embodiment, the evaporation step is undertaken at a temperature less than about 15° C. of the boiling point of the swelling agent solution, less than about 10° C. of the boiling point of the swelling agent solution, and less than about 5° C. of the boiling point of the swelling agent solution. In one embodiment, the evaporation step is undertaken at a temperature in the range of about 5° C. to about 8° C. of the boiling point of the swelling agent solution. Advantageously, the evaporation step temperature is kept below the boiling point of the swelling agent solution, which leads to a relatively slow evaporation rate of the swelling agent solution to thereby produce a void within the polymer microspheres with a conduit extending therethrough. More advantageously, because evaporation is controlled, release of swelling agent vapor flows continuously from the conduit during the evaporation step to retain the shape of the microsphere.

The evaporating step may also comprise the step of polymerizing a monomeric mixture in droplet form in the presence of the swelling agent solution to form the polymer microsphere having the swelling agent solution absorbed therein.

The disclosed embodiments also describe a method of making a micro-container comprising the steps of:

polymerizing, in droplet form, a monomeric mixture comprising a swelling agent to thereby form a polymer microsphere having the swelling agent absorbed therein; and

evaporating said swelling agent from said microsphere to form an inner void therein, wherein said evaporating is undertaken under conditions to form a conduit extending through the shell wall of the microsphere and into the inner void.

The method may comprise the step of hardening the outer shell wall before said evaporating step.

The hardening step may comprise the step of providing a cross-linking agent within said monomeric mixture.

Any cross-linking agent capable of increasing the strength of the shell wall can be used. Exemplary cross-linking agents include di-vinyl-benzene, 1,4-butane diol diacrylate, triethanolamine dimethacrylate, triethanolaminetrimethacrylate, tris (methacryloyloxymethyl) propane, allyl methacrylate, tartaric acid dimethacrylate,N, N′-methylene-bisacrylamide, hexamethylene bis (methacryloyloxyethylene) carbamate, hydroxytrimethylene dimethacrylate and 2,3-dihydroxytetramethylene dimethacrylate, 1,3-butanediol diacrylate, di (trimethylolpropane) tetraacralate, poly (ethylene glycol) diacrylate, trimethylolpropane ethoxylate, poly (propylene glycol) dimethacrylate, bisphenol A dimethacrylate and 1,4-butandiol acrylate. In one embodiment the cross-linking agent is di-vinyl-benzene (DVB) monomer.

The polymerizing step may comprise the step of providing seed particles within the swelling agent solution. The seed particles may be comprised of a polymer, optionally a relatively linear polymer or a weakly cross-linked polymer, which may be substantially non-polar. Exemplary polymers which may be used include polystyrene, polyesters, polycarbonates, polyethylenes, polypropylenes. In one embodiment, the linear polymer is polystyrene. In one embodiment, the swelling agent solution's substantially absorbed by the seed particles.

The method may comprise before said polymerizing step, the step of forming said monomeric mixture in droplet form. In one embodiment, the monomeric mixture is an emulsion. The forming step may comprise the step of forming an emulsion by providing an emulsifier into said monomeric mixture. Exemplary emulsifiers include amphiphilic organic compounds, sodium dodecyl sulfate, ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like. In one embodiment, the emulsifier is sodium dodecyl sulfate (SDS).

In one embodiment, the monomeric mixture includes polymer seed particles, a cross-linking agent, a stabilizer and an organic solvent. In one embodiment, the seed particles are substantially insoluble in said organic solvent. In one embodiment, the monomeric mixture includes monomers of styrene, DVB, methacrylate acid (MAA) and 2,2′-Azobis(2-methylpropionitrile) (AIBN).

The seed particles may be provided in a monodispersion form. Advantageously, there is substantially no mass-transfer between the monomeric mixture in droplet form during swelling. More advantageously, the use of monodispersed seeds leads to a narrow distribution of said microspheres.

The step of forming an emulsion may comprise the step of ultra-sonication. In one embodiment the ultra-sonication was carried out at room temperature for about 1-20 minutes, for about 5-15 minutes, and about 8-12 minutes. In one embodiment, the ultra-sonication was carried out for 10 minutes. In one embodiment, the resulting emulsion was magnetically stirred (400 rpm). In one embodiment, the step of stirring was carried out at room temperature for about 24 hours. In one embodiment, the emulsion is sealed with an inert gas. In one embodiment, the inert gas is nitrogen. Advantageously, the inert gas will quench the radicals from the initiators and arrest polymerization.

The step of polymerizing said monomeric mixture may comprise the step of providing a solvent that is immiscible with said swelling agent such that said monomeric mixture in droplet form is non-homogenous.

Advantageously, a phase separation of seed polymers occurs to form an interfacial polymer layer. More advantageously, the interfacial polymer later can encapsulate the monomeric mixture to create the microsphere with the inner void surrounded by the shell wall.

The step of polymerization may include free radical polymerization. Free radical polymerization may be initiated to grow macromolecules, which become progressively less soluble in the dispersed hexane/monomers mixture. Advantageously, phase separation occurs and the resulting cross-linked macromolecules may be deposited at the polar/non-polar interface. More advantageously, the deposition of the macromolecules hardens the shell wall of linear polystyrene. More advantageously, the newly formed polymers may be a site for further polymerization to further strengthen the shell wall.

The ratio of the volume of swelling agent solution (ie such as n-hexane) to the mass of seed particles may effect the morphology of the microsphere. In one embodiment, the ratio is lower than about 4 ml/mg, lower than about 3 ml/mg, lower than about 2 ml/mg, lower than about 1 ml/mg. In one embodiment, the threshold for forming said microsphere with an inner void and a conduit extending through the shell wall of the microsphere to the inner void, is between 3 ml/mg and 4 ml/mg.

The method of making a micro-container may comprise the step of providing a substance within the inner void of the microsphere. The substance may be selected from the group consisting of inks, dyes, drugs, quantum dots, catalysts. In one embodiment, the substance is a drug.

The disclosed embodiments also describe a micro-container comprising a polymer microsphere having an inner void and a conduit extending through the shell wall of the microsphere to said inner void.

The diameter of the microsphere may be in the range selected from the group consisting of: about 1 μm to about 5 μm; about 1.5 μm to about 4.5 μm; about 2 μm to about 4 μm; about 2.5 μm to about 3.5 μm. In one embodiment, the diameter of the microsphere is about 3.3 μm.

The diameter of the inner void may be in the range selected from the group consisting of: about 0.1 μm to about 4.9 μm; about 0.5 μm to about 4.5 μm; about 1 μm to about 3.5 μm; about 1.5 μm to about 3 μm; about 2.0 μm to about 2.5 μm. In one embodiment, the diameter of the inner void is about 2.3 μm.

The average thickness of the shell wall may be in the range selected from the group consisting of less than 2 μm; less than 1.5 μm; less than 1.0 μm; less than 0.5 μm. In one embodiment, the average thickness of the shell wall is 1.0 μm.

The average diameter of the conduit may be in the range selected from the group consisting of: 10 nm to 1000 nm; 50 nm to 500 nm; 100 nm to 450 nm; 150 nm to 400 nm; 200 nm to 300 nm. In one embodiment the average diameter of the conduit is 200 nm. In one embodiment, the conduit is a single pin-hole extending through the shell wall.

The outer surface of the microsphere may be modified by functional groups. Exemplary functional groups include carboxylic acid, carboxylate, sulfonate, hydroxide, alkoxide, ammonium salt and phosphate. In one embodiment, the outer surface of the microsphere is modified by carboxylate groups. Advantageously, the surface-modified carboxylate groups increases the biocompatibility of the microspheres and their water-solubility and dispersion stability in water or buffers. More advantageously, the surface-modified carboxylate groups provide anchors for bio-conjugation.

The microspheres may have more than one function. For example, they may be used to contain substances for use in drug delivery, chemical therapy, in-situ printing, catalysis, bio-imaging or in diagnostics. In one embodiment, the inner void of the microsphere can be loaded with magnetic nanoparticles and quantum dots simultaneously to perform drug delivery as well as bioimaging.

The strength of the shell wall may be governed by the relative content of the cross-linking agent in the monomeric mixture. In one embodiment, the relative content of DVB to styrene to MMA is 4:1:1. Advantageously, the relatively high material strength of the shell wall allows the microsphere to maintain its original shape without any deformation during solvent extraction.

In one embodiment, the micro-particle is biodegradable. The micro-particle may be comprised of a biodegradable polymer. Exemplary biodegradable polymers include natural polymers and their synthetic analogs, including polylactides, polysaccharides, proteoglycans, glycosaminoglycans, collagen-GAG, collagen, fibrin, and other extracellular matrix components, such as elastin, fibronectin, vitronectin, and laminin.

In one embodiment, the shell wall of the micro-particle is selectively permeable. The shell wall may be comprised of a selectively permeable polymer. The selectivity of the selectively permeable shell wall may be modified according to the molecular weight of the polymers comprising the shell wall. The polymers may be selected from the group consisting of acrylate polymers, copolymers and terpolymers such as poly(acrylic acid), poly(methacrylic acid) poly(methacrylate), poly(methyl methacrylate) and acrylate copolymers and terpolymers of acrylic acid, methacrylic acid, methacrylates, methyl methacrylates, hydroxyethyl methacrylic such as 2-hydroxyethyl methacrylate, hydroxypropylacrylate, poly(dimethylaminoethyl methacrylate) (“DMAEMA”) and copolymers and terpolymers of dimethylaminoethyl methacrylate with 2-hydroxyethyl methacrylate and/or hydroxypropylacrylate and methacrylate and/or methyl methacrylate; copolymers or terpolymers of acrylic acid and/or methacrylic acid with 2-hydroxyethyl methacrylic and/or hydroxypropylacrylate and methacrylate and/or methyl methacrylate.

The disclosed embodiments also describe a micro-container for drug delivery comprising:

a polymer microsphere having an inner void and a conduit extending through the shell wall of the microsphere to said inner void; and

a drug loaded within said inner void.

The drug may be an anti-cancer agent. Exemplary anti-cancer agents include actinomycin D, doxorubicin, daunomycin, vincristine, vinblastine, colchicine, paclitaxel, docetaxel, etoposide and hydroxyrubicin. In one embodiment the drug is doxorubicin.

Advantageously, the shell wall has a relatively high mechanical strength, which prevents the fast deformation of the microsphere and overcomes the problem of drug dumping.

The drug may be loaded within said inner void via the conduit extending through the shell wall of the microsphere into the inner void. The loading may comprise the step of agitating the microspheres in a container containing the drug. The microsphere-drug dispersion may be shaken continuously for a period selected from the group consisting of more than 2 hours, more than 10 hours, more than 20 hours, more than 30 hours, more than 40 hours. In one embodiment, the dispersion was shaken continuously for 48 hours. The drug may be loaded within said inner void by the process of simple diffusion, vacuum suction or capillary uptake.

### Morphological and Structural Characterization

The structure of the microsphere may be observed under scanning electron microscope (SEM). In one embodiment, the internal structure can be characterized by observing the cross-section of the microsphere. The microsphere may be embedded in epoxy resin, cured and further cut into thin films using a sharp scalpel for SEM characterization.

The internal structure of the microspheres may also be characterized while using fragmented microspheres. The fragmented microspheres may be prepared by crushing intact microspheres in liquid nitrogen. This is due to the sudden and fierce change in environmental temperature.

### Optical Characterization of Microsphere

The internal structure of the microsphere may be observed with the aid of confocal microscopy. The process may include the loading of an organic dye within the inner void of the microsphere. In one embodiment, Rhodamine-6G (Rd-6G) was used as a loading material into the hollow microspheres for optical characterization/observation under confocal fluorescence microscopy. Advantageously, the strong fluorescence of Rd-6G enables itself to be easily detected under fluorescence microscopy. More advantageously, it has a molecular weight of 479, which is similar to the molecular weight of most of the commonly used drugs. As such, the use of Rd-6G can help the model-building of controlled drug release.

### BRIEF DESCRIPTION OF DRAWINGS

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 is a schematic partial cut-away view of single pin-holed hollow microsphere: FIG. 1A shows the pin-holed hollow microsphere without being loaded with a substance; and FIG. 1B shows the pin-holed hollow microsphere in which various different substances are simultaneously loaded therein.

FIG. 2 shows the SEM images of single pin-holed hollow microspheres: FIG. 2A shows the SEM image of pin-holed hollow microspheres at a Magnification of ×4300; FIG. 2B shows the SEM image of pin-holed hollow microspheres at a Magnification of ×2200.

FIG. 3 shows the cross-section SEM images of single pin-holed hollow microspheres: FIG. 3A shows the cross-section SEM image of hollow microspheres at a Magnification of ×2500; FIG. 3B shows the thickness of the shells of the hollow microspheres at a Magnification of ×14000.

FIG. 4 shows the SEM image (×2000 Magnification) of the fragment of a single pin-holed hollow microsphere with a pin-hole in the internal wall of the microsphere.

FIG. 5 is a schematic representation of the process for the formation of single pin-holed hollow microspheres.

FIG. 6 shows SEM images of single pin-holed hollow microspheres: FIG. 6A reveals the morphology of hollow microspheres (×3500) with a swelling ratio of 3 ml/mg;

FIG. 6B reveals the morphology of hollow microspheres with a swelling ration of 4 ml/mg.

FIG. 7 shows confocal fluorescence images of Rd-6G-loaded single pin-holed hollow microspheres: FIG. 7A shows the cross-section image of hollow microspheres under strong fluorescence background from an unwashed Rd-6G environment; FIG. 7B show the top view image of hollow microspheres under low fluorescence background after thorough washing; and FIG. 7C shows the cross-section image of hollow microspheres under low fluorescence background after thorough washing.

FIG. 8 shows multi-step controlled release curves of DOX-loaded single pin-holed hollow microspheres in MES (pH=6.1) buffer; FIG. 8A shows the concentration of the in vitro drug (DOX) in the MES buffer during a first release process; FIG. 8B shows the concentration of the same in vitro drug (DOX) in the MES buffer during a second release process from the first release process; and FIG. 8C shows the concentration of the in vitro drug (DOX) in the MES buffer during a third release process after the second release process.

### EXAMPLES

Non-limiting examples of the invention and a comparative example will be further described in greater detail by reference to specific examples, which should not be construed as in any way limiting the scope of the invention.

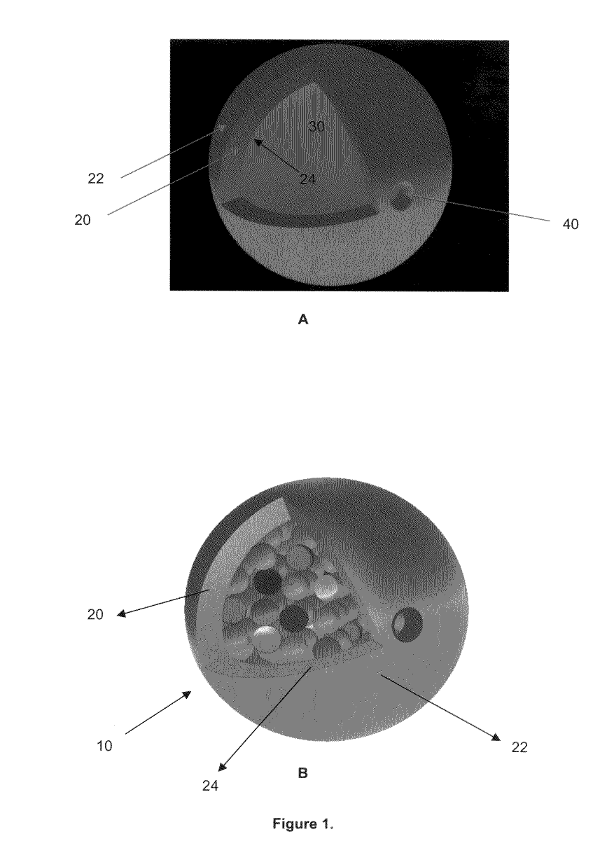

Referring to FIG. 1A, a schematic diagram in partial cut-away view of a single pin-holed hollow microsphere 10 is shown. The microsphere 10 includes a shell wall 20 having outer shell surface 22 and inner shell wall surface 24. An inner void 30 is bound by the inner shell wall 24. A single hole 40 extends through the shell wall 20 from the outer shell surface 22 to the inner shell wall surface 24. FIG. 1A is shown with the void 30 empty but FIG. 1B shows the void loaded with various different substances.

### Materials Used in the Following Examples Include:—

Unless stated otherwise, all reagents were purchased from Sigma-Aldrich Co of the United States of America:

Styrene (ST, 99%), divinylbenzene (DVB, 80% isomers), and methacrylate acid (MAA, 99%) were used after removing inhibitors.

2,2′-Azobis (2-methylpropionitrile) (AIBN) was re-crystallized from ethanol prior to use.

Poly (vinyl pyrrolidone) (PVP, MW=40,000 g/mol) and sodium dodecyl sulfate (SDS) were used as steric stabilizer and emulsifier respectively.

n-Hexane (98%), absolute ethanol, and distilled water were used throughout the experiments.

Rhodamine-6G (Rd-6G) was used as a loading material into hollow microspheres for optical characterization/observation under confocal fluorescence microscopy.

Doxorubicin hydrochloride (DOX, 99%) was incorporated into hollow microspheres for evaluating in vitro and in vivo release behaviors.

2-(N-morpholino)ethanesulfonic acid (MES) was used as a buffer for maintaining the pH of less than 6.5 so as to prevent decomposition of DOX.

### Example 1

### Preparation of Polystyrene Seed Particles

Monodispersed polystyrene seed particles were prepared in this example according to the process disclosed below.

To produce 2.2 μm polystyrene seed particles, a reaction mixture containing 1.0 ml styrene, 14 mg AIBN, 9.0 mL ethanol, 1.0 mL water and 100 mg PVP-40 were prepared by dispersion polymerization. The dispersion medium was prepared by dissolving PVP-40 into a mixture of ethanol and water in a capped culture tube. The reaction mixture was ultra-sonicated for 10 in to form a homogeneous solution. The styrene and AIBN were then added into the reaction mixture.

The reaction mixture was further degassed for 5 min with nitrogen after ultra-sonication. The reaction mixture was then sealed for preparing seed particles by shaking (150 rpm) at 70° C. for 24 hours. The obtained latex particles were washed extensively with ethanol and water by several rounds of centrifugation and decantation so as to remove the un-reacted reagents and surfactant residues. The purified polystyrene seed microspheres were freeze-dried and stored for further use.

### Example 2

### Preparation of Single Pin-Holed Hollow Microspheres

Single pin-holed hollow microspheres were prepared by using a seeded emulsion polymerization process as disclosed below.

100 μL of n-Hexane was emulsified in 10 mL of 0.25 wt % SDS aqueous solution by ultra-sonication for 10 min at room temperature. An aqueous solution (˜3.0 mL) dispersed with 100 mg polystyrene seed particles were then added into the emulsion mixture. The resulting emulsion was magnetically stirred (400 rpm) at room temperature for 24 hours for complete absorption of n-hexane by polystyrene seed particles.

Monomers of styrene (100 μL), DVB (400 μL), MAA (100 μL) and AIBN (6 mg) were mixed homogeneously and further emulsified in 10 ml of 0.25 wt % SDS aqueous solution by ultra-sonication for 10 in at room temperature. The resulting monomer emulsion was then mixed with the emulsion containing n-hexane-swollen seed particles. The mixed solution was magnetically stirred (400 rpm) at room temperature for 24 hours for a complete absorption of monomers by the n-hexane swollen seed particles.

The obtained emulsion was sealed with nitrogen and further polymerized at 60° C. for 24 hours under a continuous stirring at 400 rpm. The final products were washed extensively with ethanol and water by several rounds of centrifugation and decantation so as to remove the un-reacted reagents, surfactant residues, and small-sized particles (side product). The purified hollow microspheres were freeze-dried and stored for further use.

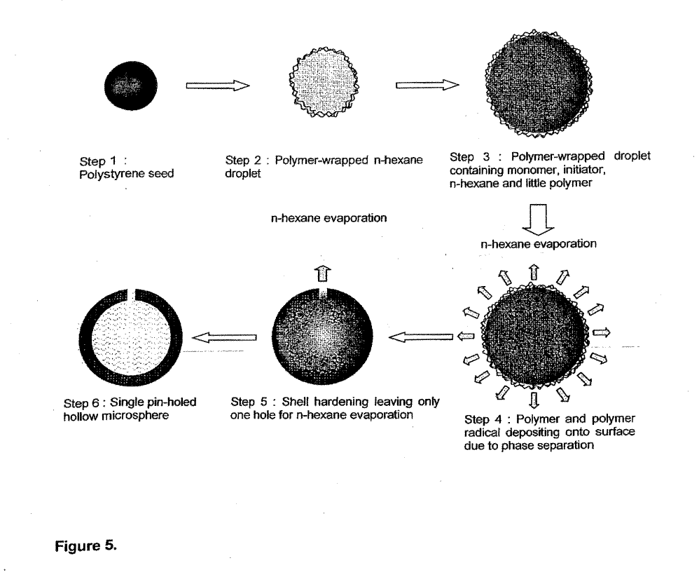

The detailed formation processes for single pin-holed hollow microspheres as described above are schematically summarised in FIG. 5 as follows:

Step 1: Polystyrene seeds were swollen by n-hexane, and phase separation occurs because n-hexane is a poor solvent for polystyrene.

Step 2: the resulting linear polystyrene molecules are thought to be wrapped around microdroplets of n-hexane as a core material, which is partially due to the lower free energy at the water-polymer interface than that at the water-n-hexane interface in this system.

Step 3: After further swelling with a mixture of monomers, a homogeneous mixture (emulsion) containing n-hexane, styrene, DVB, MAA, and AIBN in the swollen micelles. Meanwhile, seed polymers may also be dissolved in monomers as good solvents with high boiling points (145° C. for styrene and 195° C. for DVB).

Step 4: The n-hexane absorbed within the microsphere is evaporated by raising the temperature to 60° C. This causes some physical changes and chemical reactions to be initiated and to gradually proceed. Free radical polymerization can be initiated to grow macromolecules, which becomes progressively less soluble in the dispersed hexane/monomer mixtures. Phase separation occurs and the resulting crosslinked macromolecules can gradually deposit at the water-oil interface for hardening the shell of linear polystyrene. The phase separation at the water-oil interface is partially due to the polymerization with MMA to increase certain water-solubility and further push the linear polystyrene to the interface of water-oil droplets. The newly formed polymers wrapped around the core also become a site for further polymerization so as to strength linear polystyrene by forming a uniform shell.

Meanwhile, n-hexane, a low boiling point hydrocarbon (68˜69° C.) begins to evaporate and therefore the pressure in the core of the microsphere increases. The pre-formed interfacial layer of linear polystyrene by swelling seed particles can reduce the evaporation rate of hexane in order to avoid fast evaporation before forming network of DVB and styrene to harden the shell. With the use of high content of DVB in the monomer phase, the obtained highly cross-linked polymers can strengthen the shells obviously. With the formation of highly cross-linked shell and the increase in its thickness, the permeability gradually decreases (i.e. the number of pores existing for evaporation of n-hexane become less and less and the size of pores becomes smaller and smaller). The internal pressure also increases at the same time, and finally only one hole is left for out-streaming of the remained n-hexane at the weakest place on the shell (Step 5).

Step 6: Upon formation of the single hole in the microsphere container and subsequent releast of volatile substances from therein, a micro-container comprising a polymer microsphere having an inner void and a conduit extending through the shell wall of the microsphere to the inner void is created.

An important aspect of the disclosed technique involves the use of a specific swelling agent for producing single pin-holed hollow microspheres. A low evaporation rate of high-boiling point hydrocarbons at the temperature for effective polymerization (60-70° C.) only yields nonporous or macroporous shell. A proper evaporation rate of a low boiling point hydrocarbon-n-hexane at 60° C. (ie, less than the boiling point of 67-68° C. for n-hexane) leads to the formation of single pin-holed hollow microspheres by using two-stepped seeded emulsion polymerization. In contrast, no single pin-holed hollow microspheres were produced at the boiling point of hexane because all the hexane is vaporized in a short period of time (a high vapor pressure can be generated). The vapor pressure of the swelling agent should be considerably low at the temperature for polymerization (lower than boiling point), and the formation of the pin-hole relies on the continuously flowing out of the swelling agent during the polymerization to remain the shape/microstructure of polymer microspheres under constant vapor pressure. Consequently, a fast phase separation for forming strong shell before the exhaustion of hexane is also required to maintain the spherical shape under the continuous pressure of the swelling agent.

The use of high content of DVB in monomers can accelerate phase separation inside the monomeric droplets during the early stage of the polymerization due to the quick formation of highly cross-linked copolymers with reduced solubility in the monomer and solvent mixture. The strong shells can thus survive the extraction of a good solvent, THF and remain the original shape without any deformation. The effect of the relative content of DVB is further explained in the experiments of Example 6.

### Example 3

### Analysis of Microstructure of Single Pin-Holed Hollow Microspheres by Scanning Electron Microscopy

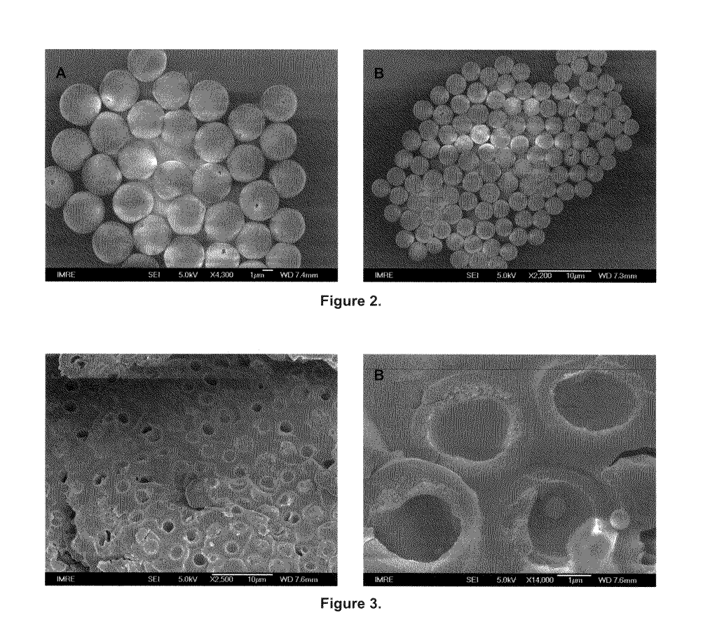

To analyze the microstructure of the single pin-holed hollow microspheres, a drop of aqueous dispersion of the hollow microspheres was dispersed on an aluminum foil and dried at room temperature for morphological and structural characterization. Uniform polymer microspheres of 3.3 μm in diameter have been clearly observed by Scanning Electron Microscopy (SEM), as shown in FIG. 2.

Referring to FIG. 2, there is a single pinhole (200-300 nm in diameter) on the surface of nearly 50% microspheres. A logical speculation is that all the microspheres should have a single pinhole because only the surface of nearly half of the microspheres can be observed under SEM. This suggests that approximately 50% pin holes were hidden in approximately 50% microspheres and were not displayed by the SEM images in FIG. 2. This speculation is to be confirmed by the incorporation of fluorescent dyes into the single pin-holed hollow microspheres as disclosed in Example 5.

### Example 4

### Characterization of the Single Pin-Holed Hollow Microspheres

The characterization of the internal structure of the single pin-holed hollow microspheres is disclosed below.

To prepare the cross-section samples of the hollow microspheres of purified single pin-holed hollow microspheres was dispersed or embedded into epoxy resin (Epoxy Embedding Medium Kit Fluka 45359). The obtained epoxy mixture was then cured and further cut into thin films using a sharp scalpel for SEM characterization. As shown in the cross-section image of single pin-holed hollow microspheres in FIG. 3, all the microspheres at the interface were cut without observable distortion in shape. The images in FIG. 3 clearly show that all of the cut particles are hollow and the average thickness of shell is slightly smaller than 1 μm. In particular, FIG. 3B shows that the shells of the microspheres are highly compact which suggest that the shells display high strength and low permeability properties.

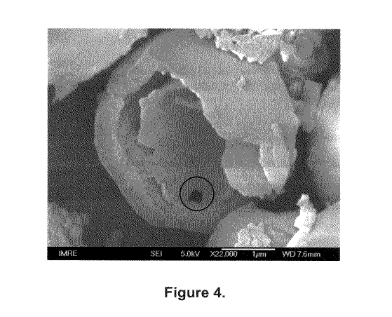

To characterize the internal structure of fragmented hollow microspheres, the microspheres were prepared by freeze-fracturing the intact hollow microspheres in liquid nitrogen. The SEM image in FIG. 4 clearly displays the hollow structure of the fragmented microspheres. Furthermore, a pin-hole with an average diameter of 200 nm can been seen from the inside of a fragmented microsphere.

The as-observed external and internal holes on the shell are actually from a tunnel in the shell (the pinhole does go through the shell of hollow microspheres, rather than a simple crater at the surface), which directly connect their internal cavities with the external. Without being bound by theory, it is speculated that the pin-holes on the inner and outer shell of the fragmented microsphere are connected by a tunnel which directly connects the internal cavity of the microsphere with the external environment. This speculation is supported by the incorporation of fluorescent dyes into the single pin-holed hollow microspheres as disclosed in Example 5.

### Example 5

### Incorporation of Rd-6G into Single Pin-Holed Hollow Microspheres for Optical Characterization

Rhodamine-6G (Rd-6G) was used as a loading material into hollow microspheres for optical characterization or observation under confocal fluorescence microscopy.

Typically, 10 mg single pin-holed hollow microspheres were well dispersed in 10 ml of saturated ethanol solution of Rd-6G. The dispersion was continuously shaken for 2 hours and then ethanol was removed. The obtained red solids were rinsed with water several times and separated by centrifugation at 7,000 rpm for 10 min. The Rd-6G-loaded hollow microspheres were re-dispersed in water for optical characterization.

The morphology and structure of the single pin-holed hollow microspheres have been extensively revealed by SEM observation. To confirm that single pin-holed hollow microspheres were prepared by the process disclosed above, the internal structure of the hollow microspheres was analyzed with the aid of confocal fluorescence microscopy.

Rd-6G was loaded into single pin-holed hollow microspheres disclosed by the process below. Rd-6G was firstly mixed with the single pin-holed hollow microspheres in ethanol for 2 hours. The obtained dispersion was then placed in a chamber, and the air inside the particles was pumped out to draw the organic dyes into the hollow microspheres. After the ethanol was completely evaporated, the organic dye solidified within the particles. The Rd-6G-loaded hollow microspheres were then rinsed with water once. The cross-section images of Rd-6G-loaded single pin-holed hollow microspheres were observed by confocal microscopy showed in FIG. 7A. In addition to the fluorescence signal from the interior of hollow microspheres, a clear fluorescence background in water was also observed due to the incomplete washing of Rd-6G in solution (green color was used for high contrast). A clear thorough hole through shell can also be seen clearly on the cross-section image of some microspheres when they have appropriate orientation.

The Rd-6G-loaded hollow microspheres were rinsed with water more than three times to remove non-specific adsorption of Rd-6G, only fluorescent microspheres were clearly observed under confocal microscope without fluorescence background, as shown in the top-view image of hollow microspheres in FIG. 7B. From the cross-section image of single pin-holed microspheres in FIG. 7C, bright, solid Rd-6G was observed clearly inside the particles, and pinholes on the shell of the microspheres were seen as well.

To measure the amount of Rd-6G loaded into the microspheres, a very intense optical spectrum of a single Rd-6G loaded particle was recorded using an excitation wavelength at 488 nm. The emission of Rd-6G reached maximum intensity at 551 nm. This evidence proves the existence of a large amount of Rd-6G inside the hollow microspheres. All the results show that a large amount of Rd-6G has been successfully loaded into the void domains of the hollow microspheres.

### Hollow Microspheres Having Low Permeability

Referring again to FIG. 7A, when the Rd-6G-loaded hollow microspheres were dispersed into solution with a strong fluorescence background, very dark shells were immediately observed because they contain an unobservable amount of dye when compared to the internal void of the particles and the external environment. The internal void of the particles and the external environment contain high concentrations of dye and therefore show strong fluorescence under a strong fluorescence background.

Referring now again to FIGS. 7B and 7C, when the Rd-6G-loaded hollow microspheres were dispersed into solution with a low fluorescence background, the shells of highly fluorescent microspheres cannot be seen clearly in the weak fluorescence background. This is due to the low dye concentrations contained within the shells of the microspheres and the background.

The above observations correspond to the results as observed by SEM in FIG. 3B and FIG. 4, in that the shell wall has a compact structure with high strength and low permeability, rather than a porous structure. As such, the microspheres are incapable of substance loading through the shell wall. However, it is apparent from FIG. 7 that all the microspheres can be loaded with fluorescent dyes through the pin-hole rather than the compact shell wall. Therefore, all the microspheres have a complete pinhole from the inside to outside of the shell for controlled loading into hollow microspheres.

Furthermore, a tunnel structure connecting the internal cavity of the microsphere and the external environment can be observed by a cross-section image of a SEM image (FIG. not shown) when an intact microsphere was accurately freeze-fractured across the holes.

### Example 6

### Characterization Tests

Different swelling ratios of n-hexane to seed particles were used in experiments to examine the effect of the volume of n-hexane on morphologies. FIG. 2 reveals the morphology of single pin-holed hollow microspheres with swelling ratio of 1 ml/mg. The size distribution was narrow and the mean diameter was around 3.3 μm. When single pin-holed hollow microspheres were prepared with the swelling ratio of 2 ml/mg, the obtained particle size and size distribution did not change much compared to the ones with the swelling ratio of 1 ml/mg, which might be caused by the faster evaporation of n-hexane.

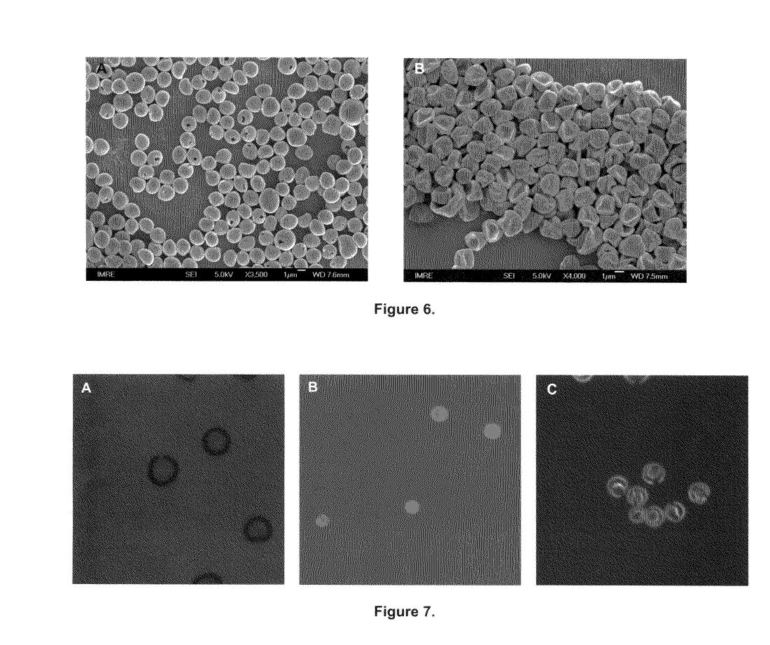

Referring to FIG. 6A, when a swelling ratio of 3 ml/mg was used, ellipsoidal microspheres with an irregular single pin-hole were prepared. However, when the swelling ratio was increased to 4 ml/mg, the shriveled football-like micro-particles without holes were obtained as can be seen in FIG. 6B.

Above the value of 4 ml/mg, the swollen seed linear polymers became much diluted by the increased volume of hexane, and there was no enough polystyrene to form interfacial layer on an increased surface area of liquid hexane droplets. It was difficult to form strong shells after polymerization due to too rapid polymerization and phase separation. The resulting micro-particles have thinner shells and were hexane-vapor-filled during the formation of polymeric micro-particles, and shriveled after complete exhaustion of absorbed hexane caused by the pressure difference between external and internal environment. It was observed that a boundary swelling ratio of between 3 ml/mg and 4 ml/mg for producing hollow microspheres with or without holes on their shells could be used. However, only when the swelling ratio is lower than the 3 ml/mg, single pin-holed microspheres can be formed.

The effect of the relative content of DVB to styrene and MMA was also investigated. When the relative content of DVB to styrene and MMA was reduced (from DVB/ST/MAA=4:1:1 to 4:2:2), a lower cross-linking degree of resulting poly(DVB-ST-MAA) was achieved accordingly. As expected, irregular microspheres were formed without pinholes. As such, the increased content of hydrophilic MAA may favor the formation of small particles in water phase.

### Example 7

### In-Vitro Release of Doxorubicin Hydrochloride (DOX)

Rd-6G was loaded into single pin-holed hollow microspheres. A range of commonly used drugs, which have a similar molecular weight of Rd-6G (479), can also be loaded therein. For example, a widely used anti-cancer drug, doxorubicin, was selected to examine its release behavior in single pin-holed hollow microspheres. Doxorubicin is usually stored in the form of doxorubicin hydrochloride (DOX, MW=580) as it is unstable at a pH of more than 6.5.

### Drug Loading

The loading procedure of DOX is identical to that of Rd-6G. Firstly, 10 mg single pin-holed hollow microspheres were dispersed in a saturated solution of DOX in ethanol (10 ml). After continuously shaking for 2 hours, the ethanol was evaporated slowly leaving DOX particles within the void. After the ethanol was completely evaporated, the organic dye solidified within the particles. After the obtained solids were rinsed by water a few times, the DOX-loaded microspheres were collected by centrifugation and further dispersed in buffer for in-vitro controlled release The concentration of DOX that is released from the microspheres was determined by a fluorescence spectrophotometer though high performance liquid chromatography (HPLC), capillary electrophoresis, UV-Vis spectrophotometer was used for this purpose.

### Drug Release

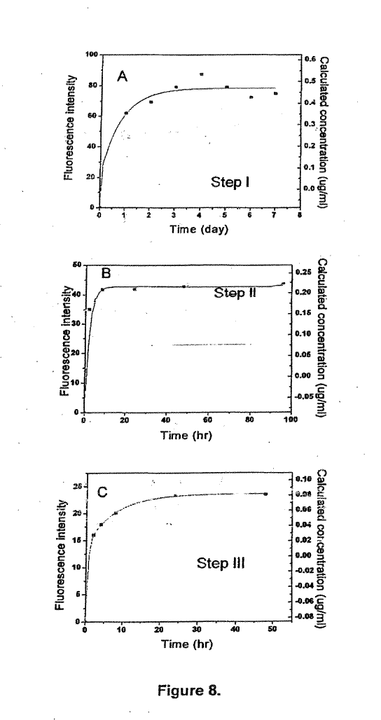

The MES buffer (pH 6.1) is used to determine the release behaviour of DOX because the buffer maintains the pH of less than 6.5 to prevent decomposition of DOX. Referring to FIG. 8, in one experiment, 1 mg hollow microspheres loaded with DOX were added in a glass cuvette containing 8 ml MES buffer. The cuvette was then shaken at 50 rpm in dark at room temperature for two days. At certain time intervals, the micro-particles were separated by centrifugation in the cuvette at 2000 rpm for 10 min as shown in FIG. 8. The DOX concentration of the supernatant in the cuvette was then determined by using a fluorescence spectrophotometer. The excitation wavelength was fixed at 480 nm; the intensities at emission wavelength of 592 nm were recorded under identical measurement condition.

Referring now to FIG. 8A, there is shown the concentration of the in vitro drug (DOX) in the MES buffer over time during a first release process; FIG. 8B shows the concentration of the same in vitro drug (DOX) in the MES buffer during a second release process from the first release process; and FIG. 8C shows the concentration of the in vitro drug (DOX) in the MES buffer during a third release process after the second release process

It can be seen from FIG. 8 that it has been found that DOX cannot be completely released in a single release process. In the first (Step I: See FIG. 8A) release process, the DOX concentration in the buffer reaches nearly equilibrium after 3 days; in the second (Step II: See FIG. 8B) and third (Step III: See FIG. 8C) process, the DOX concentration in the buffer reaches nearly equilibrium within two days

The multi-step controlled release behavior shown in FIG. 8 is explained as follows. Firstly, there is no drug consumption in the cuvette. Furthermore, the compact properties of the shell of the microspheres could not be degraded in the buffer and therefore the drug could not be completely released to the theoretical maximum. This is different from the release mechanism of microspheres using biodegradable materials, in which the release behavior is mainly controlled by the degrading speed of the shell or matrix materials. Therefore, this positively indicates that the compact shell of the micro spheres having low permeability properties allows controlled release of the drug from the pin-holed hollow microspheres.

Phosphate Buffered Saline (PBS) buffer (pH=7.4) was also chosen as the release medium due to the similar pH value (pH=7.35˜7.45) to human blood. DOX can be released to a similar level of ˜0.29 μg/m after 2-3 days, and leveled off in the subsequent 7-8 days as detected (FIG. not shown). Similarly, DOX was not released completely to the theoretical maximum. However, DOX can be further extracted by ethanol. Due to the lower surface tension and hydrophobic properties of ethanol compared to water, ethanol can easily pass through the pin-holes of the hollow microspheres to extract DOX.

### Example 8

### In-Vivo Release or Doxorubicin Hydrochloride (DOX)

### Drug Loading.

10 mg single pin-holed hollow microspheres were well dispersed in 2 ml water containing 1 mg DOX. The obtained dispersion was continuously shaken (50 rpm) for two days in the dark at room temperature. DOX-loaded hollow microspheres were separated by centrifugation at 2000 rpm for 10 min. The DOX concentrations of the supernatants were determined by using a fluorescence spectrophotometer (excitation and emission wavelengths at 480 nm and 592 nm, respectively). The standard calibration curve of different DOX concentrations with respect to the fluorescence intensities shows a linear relationship. The drug loading efficiencies are calculated according to the following equation:

E=(T−S)/T×100%

where T is the total charge (drugs for loading) of DOX; and

S is the supernatant content of DOX.

The drug loading capacitances are calculated according to the equation:

L=(T−S)/M×100%

where T is the total charge of DOX;

S is the supernatant content of DOX and M is the mass of the micro-particles.

In order to minimize the possible bio-incompatibility from other substances, only microspheres, DOX and water were used in the loading system (DOX is soluble in both water and ethanol). Drug loading experiments were carried out, and DOX was almost completely loaded into the micro-particles for each samples. The average drug loading capacitance and efficiency is ˜10.5% and ˜98%, respectively. After drying, the samples were stored in refrigerator prior to use for in vivo release. A predetermined amount of drug in microspheres (1 mg DOX and 10 mg microspheres) was prepared in convenience for quantitative loading and subsequent analysis. More DOX can be loaded using a higher drug concentration in water (A loading concentration of 0.5 mg/mL was used and much lower than its solubility of 10 mg/mL in water.).

Theoretically, the loading capacitance of single pin-holed hollow microspheres can be simply estimated by the ratio of cavity volume/total volume of hollow microspheres when assuming the densities of shell and loading materials are identical. In our case, the average diameter of internal cavity and hollow microspheres is ˜2.3 μm and 3.3 μm, respectively. Then the maximum loading capacity is >40% when the internal cavities are completely filled with solid materials.

### In Vivo Release Test.

Male Sprague Dawley rats (8 weeks, average weight 250 g) were stabilized for 1 week with free access to food and water. Microcontainers loaded with DOX were subcutaneously administered into the rats at a level of 3.7 mg/Kg (10 mg microspheres containing 1 mg DOX were freshly dispersed in 0.5 mL of normal saline solution for each rat). At predetermined time intervals as shown in FIG. 8, 0.5 mL of blood samples were withdrawn through indwelling cannulae, which were implanted in the external jugular vein of rats beforehand so as to collect the blood samples when the rats were conscious. The blood samples were separated by centrifugation at 2000 g at 4° C. for 10 min and the obtained plasma samples were then stored at −20° C. prior to analysis. Just before the DOX extraction, the plasma samples were thawed followed by adding 3 ml of 70% ethanol and 30% 0.45 M HCl aqueous solution by volume. The obtained suspensions were mixed for 30 s to form gels. These gels were first stored at 4° C. for 24 hours in dark, and then centrifuged at 10,000 g for 15 min. The supernatants were transferred into glass cuvettes and allowed to warm to room temperature for the determination of DOX concentration by a fluorescence spectrophotometer.

In comparison to in vitro release, the fluctuation of drug concentration in an animal body exhibits a behavior different from in vitro release, due to the metabolism and excretion of the drug in the animal body. In the first 8 hours, DOX concentration in blood reached to 0.4 μg/mL slowly. The DOX concentration further reached to the highest peak (1.6 μg/mL) 48 hours after the injection. Subsequently, the DOC concentration started to decrease in the following week until the drug was completely consumed.

It has always been a major goal for controlled release systems to keep the drug concentrations in the therapeutic level as long as possible. From the results disclosed above, the drug concentration was kept upon a reasonable level 0.2 μg/ml for more than 5 days. In summary, single pin-holed hollow microspheres show markedly sustained release pattern. For a comparison, the DOX concentration can be reached to a higher level of 50 μg/mL by injecting 1 mg DOX in normal saline subcutaneously (˜20 mL blood in a rat). In our case, low-dose DOX was used but can be released for a long time.

### Applications

Embodiments of the disclosed micro-containers can greatly improve the delivery and performance of drugs by controlled and targeted delivery of drugs to the active site of a patient's body.

Embodiments of the disclosed method for making the micro-containers may produce micro-containers that are relatively strong and which have a relatively narrow size distribution, thereby reducing incidence of drug dumping and incongruous release behavior of drugs in patients.

Advantageously, the disclosed micro-containers may be used as both drug encapsulants and vehicles of drug carriage, in which the active agent is protected during its passage through the body or in storage until its release or undergoes controlled release within the body.

Advantageously, the disclosed method for making micro-containers may avoid the problems associated with synthesizing hollow polymer microspheres which can make in-situ encapsulation of sensitive materials impossible.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of making a micro-container comprising the step of evaporating a swelling agent solution absorbed in a polymer micro-particle to form an inner void therein, wherein said evaporating is undertaken under conditions to form a conduit extending through the shell wall of said micro-particle and into the inner void.

2. The method of claim 1, wherein said micro-particle is a microsphere.

3. The method of claim 1, comprising the step of selecting an organic liquid as said swelling agent solution.

4. The method of claim 3, wherein the organic liquid is a non-polar hydrocarbon liquid.

5. The method of claim 4, wherein the non-polar hydrocarbon liquid is selected from the group consisting of n-pentane, n-hexane, n-heptane and n-octane.

6. The method of claim 1, wherein the evaporating step is undertaken at a temperature less than the boiling point of the swelling agent solution, or at a temperature less than 15° C. of the boiling point of the swelling agent solution, or at a temperature less than 10° C. of the boiling point of the swelling agent solution.

7. The method of claim 1, wherein the evaporating step comprises the step of polymerizing a monomeric mixture in droplet form in the presence of the swelling agent solution to form the micro-particle in the form of a polymer microsphere having the swelling agent solution absorbed therein.

8. The method of claim 1, comprising the step of hardening the outer shell wall.

9. The method of claim 8, wherein the hardening step comprises the step of providing a cross-linking agent within said monomeric mixture.

10. The method of claim 7, wherein the polymerizing step comprises the step of providing seed particles within the swelling agent solution.

11. The method of claim 10, wherein the seed particles are comprised of a polymer that is substantially non-polar.

12. The method of claim 7, wherein the monomeric mixture is in the form of an emulsion of monomers.

13. The method of claim 7, wherein the monomeric mixture includes polymer seed particles, a cross-linking agent, a stabilizer and an organic solvent.

14. The method of claim 10, wherein the seed particles are provided in a monodispersion form.

15. The method of claim 7, wherein the polymerizing step comprises the step of providing a solvent that is immiscible with said swelling agent solution.

16. The method of claim 10, wherein the volume ratio of swelling agent solution to the mass of seed particles is lower than about 3 ml/mg.

17. The method of claim 1, comprising the step of loading said inner void with a substance.

18. The method of claim 17, wherein the substance is selected from the group consisting of inks, dyes, drugs, quantum dots and catalysts.

19. The method of claim 17, wherein the loading step comprises loading said inner void with a drug dissolved in a solvent.

20. The method of claim 19, comprising the step of evaporating the solvent to form drug particles in said void.

21. A micro-container comprising a polymer micro-particle having an inner void and a conduit extending through the shell wall of the micro-particle to said inner void.

22. The micro-container of claim 23, wherein said micro-particle is in the form of a microsphere.

23. The micro-container of claim 21, wherein the diameter of the micro-particle is in the range of 1 μm to 5 μm.

24. The micro-container of claim 21, wherein the diameter of the inner void is in the range of 0.1 μm to 4.9 μm.

25. The micro-container of claim 21, wherein the average thickness of the shell wall is less than about 2 μm.

26. The micro-container of claim 21, wherein the average diameter of the conduit is in the range of 50 nm to 500 nm.

27. The micro-container of claim 21, wherein the outer surface of the micro-particle is chemically modified by a functional group.

28. The micro-container of claim 27, wherein the functional group is carboxylate.

29. The micro-container of claim 21, wherein the micro-particle is biodegradable.

30. The micro-container of claim 21, wherein the shell wall is selectively permeable.
